SEQUENCE LISTING (15 sequences)

SEQ ID NO: 1 - protein, 1119 aa, synthetic construct
MARSPGRAYALLLLLICFNVGSGLHLQVLSTRNENKLLPKHPHLVRQKRAWITAPVALREGEDLSKKNPIAKIHSDLAEERGLKITYKYTGKGITEPPFGIFVFNKDTGELNVTSILDREETPFFLLTGYALDARGNNVEKPLELRIKVLDINDNEPVFTQDVFVGSVEELSAAHTLVMKINATDADEPNTLNSKISYRIVSLEPAYPPVFYLNKDTGEIYTTSVTLDREEHSSYTLTVEARDGNGEVTDKPVKQAQVQIRILDVNDNIPVVENKVLEGMVEENQVNVEVTRIKVFDADEIGSDNWLANFTFASGNEGGYFHIETDAQTNEGIVTLIKEVDYEEMKNLDFSVIVANKAAFHKSIRSKYKPTPIPIKVKVKNVKEGIHFKSSVISIYVSESMDRSSKGQIIGNFQAFDEDTGLPAHARYVKLEDRDNWISVDSVTSEIKLAKLPDFESRYVQNGTYTVKIVAISEDYPRKTITGTVLINVEDINDNCPTLIEPVQTICHDAEYVNVTAEDLDGHPNSGPFSFSVIDKPPGMAEKWKIARQESTSVLLQQSEKKLGRSEIQFLISDNQGFSCPEKQVLTLTVCECLHGSGCREAQHDSYVGLGPAAIALMILAFLLLLLVPLLLLMCHCGKGAKGFTPIPGTIEMLHPWNNEGAPPEDKVVPSFLPVDQGGSLVGRNGVGGMAKEATMKGSSSASIVKGQHEMSEMDGRWEEHRSLLSGRATQFTGATGAIMTTETTKTARATGASRDMAGAQAAAVALNEEFLRNYFTDKAASYTEEDENHTAKDCLLVYSQEETESLNASIGCCSFIEGELDDRFLDDLGLKFKTLAEVCLGQKIDINKEIEQRQKPATETSMNTASHSLCEQTMVNSENTYSSGSSFPVPKSLQEANAEKVTQEIVTERSVSSRQAQKVATPLPDPMASRNVIATETSYVTGSTMPPTTVILGPSQPQSLIVTERVYAPASTLVDQPYANEGTVVVTERVIQPHGGGSNPLEGTQHLQDVPYVMVRERESFLAPSSGVQPTLAMPNIAVGQNVTVTERVLAPASTLQSSYQIPTENSMTARNTTVSGAGVPGPLPDFGLEESGHSNSTITTSSTRVTKHSTVQHSYSP

SEQ ID NO: 2 - DNA, 3357 bp, synthetic construct
  source 1..3357: mol_type = other DNA
atggcgcggagcccgggacgcgcgtacgccctgctgcttctcctgatctgctttaacgttggaagtggacttcacttacaggtcttaagcacaagaaatgaaaataagctgcttcctaaacatcctcatttagtgcggcaaaagcgcgcctggatcaccgcccccgtggctcttcgggagggagaggatctgtccaagaagaatccaattgccaagatacattctgatcttgcagaagaaagaggactcaaaattacttacaaatacactggaaaagggattacagagccaccttttggtatatttgtctttaacaaagatactggagaactgaatgttaccagcattcttgatcgagaagaaacaccattttttctgctaacaggttacgctttggatgcaagaggaaacaatgtagagaaacccttagagctacgcattaaggttcttgatatcaatgacaacgaaccagtgttcacacaggatgtctttgttgggtctgttgaagagttgagtgcagcacatactcttgtgatgaaaatcaatgcaacagatgcagatgagcccaataccctgaattcgaaaatttcctatagaatcgtatctctggagcctgcttatcctccagtgttctacctaaataaagatacaggagagatttatacaaccagtgttaccttggacagagaggaacacagcagctacactttgacagtagaagcaagagatggcaatggagaagttacagacaaacctgtaaaacaagctcaagttcagattcgtattttggatgtcaatgacaatatacctgtagtagaaaataaagtgcttgaagggatggttgaagaaaatcaagtcaatgtagaagttacgcgcataaaagtgttcgatgcagatgaaataggttctgataattggctggcaaattttacatttgcatcaggaaatgaaggaggttatttccacatagaaacagatgctcaaactaacgaaggaattgtgacccttattaaggaagtagattatgaagaaatgaagaatcttgacttcagtgttattgtcgctaataaagcagcttttcacaagtcgattaggagtaaatacaagcctacacccattcccatcaaggtcaaagtgaaaaatgtgaaagaaggcattcattttaaaagcagcgtcatctcaatttatgttagcgagagcatggatagatcaagcaaaggccaaataattggaaattttcaagcttttgatgaggacactggactaccagcccatgcaagatatgtaaaattagaagatagagataattggatctctgtggattctgtcacatctgaaattaaacttgcaaaacttcctgattttgaatctagatatgttcaaaatggcacatacactgtaaagattgtggccatatcagaagattatcctagaaaaaccatcactggcacagtccttatcaatgttgaagacatcaacgacaactgtcccacactgatagagcctgtgcagacaatctgtcacgatgcagagtatgtgaatgttactgcagaggacctggatggacacccaaacagtggccctttcagtttctccgtcattgacaaaccacctggcatggcagaaaaatggaaaatagcacgccaagaaagtaccagtgtgctgctgcaacaaagtgagaaaaagcttgggagaagtgaaattcagttcctgatttcagacaatcagggttttagttgtcctgaaaagcaggtccttacactcacagtttgtgagtgtctgcatggcagcggctgcagggaagcacagcatgactcctatgtgggcctgggacccgcagcaattgcgctcatgattttggcctttctgctcctgctattggtaccacttttactgctgatgtgccattgcggaaagggcgccaaaggctttacccccatacctggcaccatagagatgctgcatccttggaataatgaaggagcaccacctgaagacaaggtggtgccatcatttctgccagtggatcaagggggcagtctagtaggaagaaatggagtaggaggtatggccaaggaagccacgatgaaaggaagtagctctgcttccattgtcaaagggcaacatgagatgtccgagatggatggaaggtgggaagaacacagaagcctgctttctggtagagctacccagtttacaggggccacaggcgctatcatgaccactgaaaccacgaagaccgcaagggccacaggggcttccagagacatggccggagctcaggcagctgctgttgcactgaacgaagaattcttaagaaattatttcactgataaagcggcctcttacactgaggaagatgaaaatcacacagccaaagattgccttctggtttattctcaggaagaaactgaatcgctgaatgcttctattggttgttgcagttttattgaaggagagctagatgaccgcttcttagatgatttgggacttaaattcaagacgctagctgaagtttgcctgggtcaaaaaatagatataaataaggaaattgagcagagacaaaaacctgccacagaaacaagtatgaacacagcttcacattcactctgtgagcaaactatggttaattcagagaatacctactcctctggcagtagcttcccagttccaaaatctttgcaagaagccaatgcagagaaagtaactcaggaaatagtcactgaaagatctgtgtcttctaggcaggcgcaaaaggtagctacacctcttcctgacccaatggcttctagaaatgtgatagcaacagaaacttcctatgtcacagggtccactatgccaccaaccactgtgatcctgggtcctagccagccacagagccttattgtgacagagagggtgtatgctccagcttctaccttggtagatcagccttatgctaatgaaggtacagttgtggtcactgaaagagtaatacagcctcatgggggtggatcgaatcctctggaaggcactcagcatcttcaagatgtaccttacgtcatggtgagggaaagagagagcttccttgcccccagctcaggtgtgcagcctactctggccatgcctaatatagcagtaggacagaatgtgacagtgacagaaagagttctagcacctgcttccactctgcaatccagttaccagattcccactgaaaattctatgacggctaggaacaccacggtgtctggagctggagtccctggccctctgccagattttggtttagaggaatctggtcattctaattctaccataaccacatcttccaccagagtcaccaagcatagcactgtacagcattcttactccccg

SEQ ID NO: 3 - protein, 75 aa, synthetic construct
MKRARPSEDTFNPVYPYDTETGPPTVPFLTPPFVSPNGFQESPPGVLSLRLSEPLVTSNGMLALKMGNGLSLDEA

SEQ ID NO: 4 - protein, 324 aa, synthetic construct
GNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKL

SEQ ID NO: 5 - protein, 188 aa, synthetic construct
TLWTGPKPEANCIIEYGKQNPDSKLTLILVKNGGIVNGYVTLMGASDYVNTLFKNKNVSINVELYFDATGHILPDSSSLKTDLELKYKQTADFSARGFMPSTTAYPFVLPNAGTHNENYIFGQCYYKASDGALFPLEVTVMLNKRLPDSRTSYVMTFLWSLNAGLAPETTQATLITSPFTFSYIREDD

SEQ ID NO: 6 - protein, 587 aa, synthetic construct
MKRARPSEDTFNPVYPYDTETGPPTVPFLTPPFVSPNGFQESPPGVLSLRLSEPLVTSNGMLALKMGNGLSLDEAGNLTSQNVTTVSPPLKKTKSNINLEISAPLTVTSEALTVAAAAPLMVAGNTLTMQSQAPLTVHDSKLSIATQGPLTVSEGKLALQTSGPLTTTDSSTLTITASPPLTTATGSLGIDLKEPIYTQNGKLGLKYGAPLHVTDDLNTLTVATGPGVTINNTSLQTKVTGALGFDSQGNMQLNVAGGLRIDSQNRRLILDVSYPFDAQNQLNLRLGQGPLFINSAHNLDINYNKGLYLFTASNNSKKLEVNLSTAKGLMFDATAIAINAGDGLEFGSPNAPNTNPLKTKIGHGLEFDSNKAMVPKLGTGLSFDSTGAITVGNKNNDKLTLWTGPKPEANCIIEYGKQNPDSKLTLILVKNGGIVNGYVTLMGASDYVNTLFKNKNVSINVELYFDATGHILPDSSSLKTDLELKYKQTADFSARGFMPSTTAYPFVLPNAGTHNENYIFGQCYYKASDGALFPLEVTVMLNKRLPDSRTSYVMTFLWSLNAGLAPETTQATLITSPFTFSYIREDD

SEQ ID NO: 7 - DNA, 225 bp, synthetic construct
  source 1..225: mol_type = other DNA
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggcc

SEQ ID NO: 8 - DNA, 972 bp, synthetic construct
  source 1..972: mol_type = other DNA
ggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagcta

SEQ ID NO: 9 - DNA, 567 bp, synthetic construct
  source 1..567: mol_type = other DNA
accctatggacaggtccaaaaccagaagccaactgcataattgaatacgggaaacaaaacccagatagcaaactaactttaatccttgtaaaaaatggaggaattgttaatggatatgtaacgctaatgggagcctcagactacgttaacaccttatttaaaaacaaaaatgtctccattaatgtagaactatactttgatgccactggtcatatattaccagactcatcttctcttaaaacagatctagaactaaaatacaagcaaaccgctgactttagtgcaagaggttttatgccaagtactacagcgtatccatttgtccttcctaatgcgggaacacataatgaaaattatatttttggtcaatgctactacaaagcaagcgatggtgccctttttccgttggaagttactgttatgcttaataaacgcctgccagatagtcgcacatcctatgttatgacttttttatggtccttgaatgctggtctagctccagaaactactcaggcaaccctcataacctccccatttaccttttcctatattagagaagatgactaa

SEQ ID NO: 10 - DNA, 1764 bp, synthetic construct
  source 1..1764: mol_type = other DNA
atgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaaccctatggacaggtccaaaaccagaagccaactgcataattgaatacgggaaacaaaacccagatagcaaactaactttaatccttgtaaaaaatggaggaattgttaatggatatgtaacgctaatgggagcctcagactacgttaacaccttatttaaaaacaaaaatgtctccattaatgtagaactatactttgatgccactggtcatatattaccagactcatcttctcttaaaacagatctagaactaaaatacaagcaaaccgctgactttagtgcaagaggttttatgccaagtactacagcgtatccatttgtccttcctaatgcgggaacacataatgaaaattatatttttggtcaatgctactacaaagcaagcgatggtgccctttttccgttggaagttactgttatgcttaataaacgcctgccagatagtcgcacatcctatgttatgacttttttatggtccttgaatgctggtctagctccagaaactactcaggcaaccctcataacctccccatttaccttttcctatattagagaagatgactaa

SEQ ID NO: 11 - DNA, 455 bp, synthetic construct
  source 1..455: mol_type = other DNA
tggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgctccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcg

SEQ ID NO: 12 - DNA, 900 bp, synthetic construct
  source 1..900: mol_type = other DNA
acaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtga

SEQ ID NO: 13 - DNA, 1797 bp, synthetic construct
  source 1..1797: mol_type = other DNA
catggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattga

SEQ ID NO: 14 - DNA, 572 bp, synthetic construct
  source 1..572: mol_type = other DNA
cccctctccctcccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataa

SEQ ID NO: 15 - DNA, 45036 bp, synthetic construct
  source 1..45036: mol_type = other DNA
taacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggagctagcaataaaatatctttattttcattacatctgtgtgttggttttttgtgtggctagcactagttggcccctccctcgggttaccccacagcctaggccgattcgacctctctccgctggggccctcgctggcgtccctgcaccctgggagcgcgagcggcgcgcgggcggggaagcgcggcccagacccccgggtccgcccggagcagctgcgctgtcggggccaggccgggctcccagtggattcgcgggcacagacgcccaggaccgcgctccccacgtggcggagggactggggacccgggcacccgtcctgccccttcaccttccagctccgcctcctccgcgcggaccccgccccgtcccgacccctcccgggtccccggcccagccccctccgggccctcccagcccctccccttcctttccgcggccccgccctctcctcgcggcgcgagtttcaggcagcgctgcgtcctgctgcgcacgtgggaagccctggccccggccacccccgcgactagtacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgacccctctccctcccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccgggatcctctagagtcgacatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaagtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaacggtccgggccagagtggccaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccggctgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgaacctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggcacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgagcagctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgccgttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaacagaaattaatcatgcagctgggagagtcctaaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcagccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggctagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactataacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccagccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacacgcggcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgcctaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgttgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatacgcgcccaccgaaaccgaattctcctggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgctcttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgaggccaatgaggccatgttaagtggagaggcagagcaactgcgcctgaaacacctggtccactgtcgccgccacaagtgctttgcccgcgactccggtgagttttgctactttgaattgcccgaggatcatatcgagggcccggcgcacggcgtccggcttaccgcccagggagagcttgcccgtagcctgattcgggagtttacccagcgccccctgctagttgagcgggacaggggaccctgtgttctcactgtgatttgcaactgtcctaaccctggattacatcaagatctcctaggctagcactagtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccggccgccaccatgactgccctgaccgaaggtgctaagctgtttgagaaggagattccgtacatcaccgagctggaaggggacgtcgaaggaatgaagttcatcatcaagggagaaggaaccggggacgctacgactggaaccattaaggccaagtatatctgtaccactggagatctgccagtgccttgggccacccttgtgtcaaccctctcgtatggagtgcagtgttttgctaagtaccctagccacattaaggacttcttcaaatccgccatgccggaaggttatacccaagagcgcaccatttcttttgagggagatggagtgtacaagacccgcgcgatggtcacctatgagaggggatctatctacaaccgggtgactctgactggagaaaactttaagaaggacgggcatattcttcggaagaatgtcgccttccagtgccctcccagcatcctttacattctccccgacactgtgaacaacggaatccgcgtggagttcaatcaagcctacgacatcgagggggtgacggagaagctggtgaccaagtgtagccagatgaatcggccactggccggttcagcggctgtccacattccgcgctaccatcatatcacttatcacactaagctctccaaagaccgcgatgagaggagagatcacatgtgcctggtggaagtggtcaaggccgtcgatctcgatacctatcagtaaaacttgtttattgcagcttataatggttacaaataaagcaatagcatcacaaatttcacaaataaagcatttttttcactgcattctagttgtggtttgtccaaactcatcaatgtatcttaactagttctagacaattgactctatgtgggatatgctccagcgctacaaccttgaagtcaggcttcctggatgtcagcatctgactttggccagcacctgtcccgcggatttgttccagtccaactacagcgacccaccctaacagagatgaccaacacaaccaacgcggccgccgctaccggacttacatctaccacaaatacaccccaagtttctgcctttgtcaataactgggataacttgggcatgtggtggttctccatagcgcttatgtttgtatgccttattattatgtggctcatctgctgcctaaagcgcaaacgcgcccgaccacccatctatagtcccatcattgtgctacacccaaacaatgatggaatccatagattggacggactgaaacacatgttcttttctcttacagtatgattaaatgagacatgattcctcgagtttttatattactgacccttgttgcgcttttttgtgcgtgctccacattggctgcggtttctcacatcgaagtagactgcattccagccttcacagtctatttgctttacggatttgtcaccctcacgctcatctgcagcctcatcactgtggtcatcgcctttatccagtgcattgactgggtctgtgtgcgctttgcatatctcagacaccatccccagtacagggacaggactatagctgagcttcttagaattctttaattatgaaatttactgtgacttttctgctgattatttgcaccctatctgcgttttgttccccgacctccaagcctcaaagacatatatcatgcagattcactcgtatatggaatattccaagttgctacaatgaaaaaagcgatctttccgaagcctggttatatgcaatcatctctgttatggtgttctgcagtaccatcttagccctagctatatatccctaccttgacattggctggaacgcaatagatgccatgaaccacccaactttccccgcgcccgctatgcttccactgcaacaagttgttgccggcggctttgtcccagccaatcagcctcgcccaccttctcccacccccactgaaatcagctactttaatctaacaggaggagatgactgacaccctagatctagaaatggacggaattattacagagcagcgcctgctagaaagacgcagggcagcggccgagcaacagcgcatgaatcaagagctccaagacatggttaacttgcaccagtgcaaaaggggtatcttttgtctggtaaagcaggccaaagtcacctacgacagtaataccaccggacaccgccttagctacaagttgccaaccaagcgtcagaaattggtggtcatggtgggagaaaagcccattaccataactcagcactcggtagaaaccgaaggctgcattcactcaccttgtcaaggacctgaggatctctgcacccttattaagaccctgtgcggtctcaaagatcttattccctttaactaataaaaaaaaataataaagcatcacttacttaaaatcagttagcaaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaaccctatggacaggtccaaaaccagaagccaactgcataattgaatacgggaaacaaaacccagatagcaaactaactttaatccttgtaaaaaatggaggaattgttaatggatatgtaacgctaatgggagcctcagactacgttaacaccttatttaaaaacaaaaatgtctccattaatgtagaactatactttgatgccactggtcatatattaccagactcatcttctcttaaaacagatctagaactaaaatacaagcaaaccgctgactttagtgcaagaggttttatgccaagtactacagcgtatccatttgtccttcctaatgcgggaacacataatgaaaattatatttttggtcaatgctactacaaagcaagcgatggtgccctttttccgttggaagttactgttatgcttaataaacgcctgccagatagtcgcacatcctatgttatgacttttttatggtccttgaatgctggtctagctccagaaactactcaggcaaccctcataacctccccatttaccttttcctatattagagaagatgactaataaagaatcgtttgtgggccaccatggccttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaatttaaatatcgcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgttgggtttcgccacctctgacttgagcgtcgatttttgtgatgctcgtcaggggggcggagcctatggaaaaacgccagcaacgcggcctttttacggttcctggccttttgctggccttttgctcacatgtacggggagttctggctttcggtgccagcatctgcgccacaccaccgagcaccatactggcaccgagagaaaacaggatgccggtcataccaccggccccaatggctgccccccatgctgcaagggtggctccggcggtaaagaatgatccggcaatggcggcagcccccaggacaatctggaatacgccacctgacttggccccggcgactctgggaacaatatgaattacagcgccatcaggcagagtctcatgtaactgcgccgttaacccggacgtgctgacgtcccgcccggcaatccgtacctgataccagccgtcgctcagtttctgacgaaacgccgggagctgtgtggccagtgcccggatggcttcagcccccgttttcacacgaaggtcgatgcggcgaccaaatcgttgtaaatccccgtaaaggcagatgcgcgcgcgccatgcccggtgacgccagagggagtgtgtgcgtcgctgccatttgtcggtgtacctctctcgtttgctcagttgttcaggaatatggtgcagcagctcgccgtcgccgcagtaaattgcggcgtgattcggcactgatgaaccaaaacagcacagcagcacatcgcccggctgtgccgctgacaacggcacctgatacagccccgtcgcctccagattatccagatagagattctggccgttacgccaccagtcatcctcacgatgaaagtccggcatctcaatccccgccagatgataagcatcccggaacagtgtgtaacagtccgtcaccgaaaactcctccagtctgctggcgttatcctccgtcagctcgactttttccagcagctccttgccgagatgggattcggttatcttgcctttgaaaaaatccaggtaaccttccgcatcatcgctcgcccgaccgacggcctccacgaatgccgatttgccaacggtgttcacactgcggatataaaagtaataatcatggcccggtttgatattgatactggcggctatccagtacagcgccgtaccaagataacgcgtgctggtttcaacctgtctgatatccgcaatctgcttttccgagaaccagaactcaaactgtaccgtcgggtcataaacggcaagatgcggcgtggcggttatctgaaaatagcccggcgtcagctcaatcctcgacggtgctgccggtgcggcaatccggaacgataccgacgccggatcgccctgctgcccccacgcatttaccgcccggactgtcagcctgtagttccccagcgccagttgcgtgaagcggtatgtggtttccgtcgtccgggccgtgctgaccagccgctcactgccgtcgtccgctgttacggtcagacggagcaggaaactcacgcccttcaccaccttcggtgtgtcccatcgcgccagcacctgatattccccgctgtctgcagtgacttctgcggtcaggtgctgcaccgctggcggcgtgacaccattcaccgtgccactctgttcgccgtcaaagtgcgccccgttatccacgatggcctctttttccggcacatgctgcacggcggtgatggcatacgtgccgtcgtcgttctcacggatactcacgcagcggaacagtcgctggcgcagcgtcggcagcttcagctcccatacgctgtattcagcaacaccgtcaggaacacggctcacttttaccttcacgccgtcggtgacggactgaacctccacgctgaccggattgccacttccgtcaaccaggcttatcagcgcggtaccggaggatggcagcgtgatttcacggtcgagcgtcagcgtccgggtctggctgttcaccgccagcacacgaccaccggtgctgataccggcatagtcatcatcgcagatttcaataacatcgcccggtacatggcgaagcccttctgcgccgacgctgaaatccacggtctgcgtttccagcagttctgttttaatcagccacagcccggcgcggtgtgcctgcccccggctggtacagccaaaggcatccatcttcgtaacattacgaccgtaacgggcaatggcctgcgtatcttcaacaagctctgtcgccgtctcccagccgttgttcgggtcaatccagttcacctcaacggcattatggcggtccttcagggcgctgaagctgtagcggaacggcgcggaccgctataaccgcactcgtcaccgcgataggtccaggtgcaggtgttggccagcatgatacgtcccggaaaaacagcgccatccgtttccgtcggcgtggacagtacaaaggaggcactcaccgcgctcagttcgctgcactgctcaatgcgccagcggctgatcacctcctgctccggatcggcgtaactgtttccgttgacgaagttcaccgcatccagaaaacgggcgtaaaccttacgccggaccaccgttccgccgaccagactctgcatatcttccgccatcccggtgaccataccgtacaggttagaaaccgtcagcgtggggcgcgtactggtgcctttgccattcagttcaaaaccgctcccctgaatgggatacggctgatactgtcgcccctgccaggtgaccggctcacctttttcgttctgctcattacagaaaaaataacgttctccaccgacctctggtcaggtcgatttcccagagcaccacgctggccgactgctccgcacgggtgcattcattcagtgtttcctgccggatatcctgcatcagttcaccacctgttcaaactctgcgctgaactcaacacgcagcatactgacccgcgacgaccattttgcgcaggtcacctttatctgccgccactcataaggcggcgtccacagaaaggatttccagcccccgtgctcttccagaaacgactccagtaccgtggcctcctcacgggggacagaaagcgtcacgctgtacgttttcaggttggcattcagcccggcaggcgctcgctgagaatagccatcaccaaagcgcacctttcttacagaagggaccgaagccacatccataccgggtttcactttccagcggaaggtcttcatcgtccacctccggagaacaggccaccatcacgcatctgtgtctgaatttcatcacgggcacccttgcgggccatgtcatacaccgccttcagagcagccggacctatctgcccgttcgtgccgtcgttgttaatcaccacatggttattctgctcaaacgtcccggacgcctgcgaccggctgtctgccatgctgcccggtgtaccgacataaccgccggtggcatagccgcgcatcagccggtaaagattccccacgccaatccggctggttgcctccttcgtgaagacaaactcaccacggtgaacaatccccgctggctcatatttgccgccggttcccgtaaatcctccggttgcaaaatggaatttcgccgcagcggcctgaatggctgtaccgcctgacgcggatgcgccgccaccaacagccccgccaatggcgctgccgatactcccgacaatccccaccattgcctgcttaagcagaatttctgtcatcatggacagcacggaacgggtgaagctgcgccagttctgctcactgccggtcagcatcgccgccatattctgtgcaataccatcaaaggtctgcgtggctgcactttttacctgcgacatactgtccgtggcgctctcttcccactcactccagccggacttcaggcctgccatccagttcccgcgaagctggtcttcagccgcccaggtctttttctgctctgacatgacgttattcagcgccagcggattatcgccatactgttccttcaggcgctgttccgtggcttcccgttctgcctgccggtcagtcagcccccggcttttcgcatcaatggcggcccgttttgcccgttgctgctgtgcgaatttatccgcctgctgcgccagcgcgttcaggcgctcctgatacgtaaccttgtcgccaagtgcagccagctggcgtttgtactccagcgtctcatctttatgcgccagcagggatttctcctgtgcagacagctggcgacgttgcgccgcctcctccagtaccgcgaactgactctccgccttccacaaatcccggcgctgctggctgattttctcatttgctccggcatgcttctccagcgtccggagttctgcctgaagcgtcagcagggcagcatgagcactgtcttcctgacgatcgcccgcagacaccttcacgctggactgtttcggctttttcagcgtcgcttcataatcctttttcgccgccgccatcagcgtgttgtaatccgcctgcaggattttcccgtctttcagtgccttgttcagttcttcctgacgggcggtatatttctccagcggcgtctgcagccgttcgtaagccttctgcgcctcttcggtatatttcagccgtgacgcttcggtatcgctctgctgctgcgcatttttgtcctgttgagtctgctgctcagccttctttcgggcggcttcaagcgcaagacgggccttttcacgatcatcccagtaacgcgcccgcgcttcatcgttaacaaaataatcatccttgcgcagattccagatgtcgtctgctttcttatacgcagcctctgccttaatcagcatctcctgcgcggtatcaggacgaccaatatccagcaccgcatcccacatggatttgaatgcccgcgcagtcctgtctgcccaggtctccagcgtgcccatgttctctttcaggcggcgggtctggtcatcaaaccctttcgttgcggcctcgttcgccgcctgcaatgccccggcttcatcgccggaacgctgcaactgagcaacatacgcaatctgctccgccgacacgttatggaactggcgagccatcgccgtcagccccgacgtcgggtctgtggtcagcttcccgaaggcttcagcgacctttgtccacctccacgccggatgcagaggagaaacgcgccacgatcttctagaggcctagaaggccagagtggcagagactgcattcgaaaacgtttgaattgataattattatcatttgcgggtcaattcttagaaaaactcatcgagcatcaaatgaaactgcaatttattcatatcaggattatcaataccatatttttgaaaaagccgtttctgtaatgaaggagaaaactcaccgaggcagttccataggattgcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgcgaaaacgtttgaattgataattattatcatttgcgggtcctttccggcgatccgccttgttacggggcggcgacctcgcgggttttcgctatttatgaaaattttccggtttaaggcgtttccgttcttcttcgtcataacttaatgtttttatttaaaataccctctgaaaagaaaggaaacgacagctgaaagcgagctttttggcctctgtcgtttcctttctctgtttttgtccgtggaatgaacaatggaagtcggcctcgtgatacgcctatttttataggttaatgtcatgataataatggtttcttagcgatatttaaattaat

(19) United States
(12) Patent Application Publication
(10) Pub. No.: US 2025/0188426 A1
(43) Pub. Date: Jun. 12, 2025

(54) ADENOVIRUS TYPE 5/3 FOR DETECTING CIRCULATING TUMOR CELLS

(71) Applicant: CURIGIN CO., LTD., Seoul (KR)

(72) Inventors: Jin Woo CHOI, Seoul (KR); Jae Won SONG, Seoul (KR)

(21) Appl. No.: 18/843,814

(22) PCT Filed: Jan. 13, 2023
(86) PCT No.: PCT/KR2023/000671

§ 371 Date: Sep. 4, 2024

(30) Foreign Application Priority Data
Mar. 4, 2022 (KR) 10-2022-0027832

(51) Int. Cl. C12N 7/00; C12Q 1/6886

(52) U.S. Cl. CPC C12N 7/00; C12Q 1/6886; C12N 2710/10031; C12N 2840/203

(57) ABSTRACT

The present disclosure relates to a chimeric adenovirus for detecting circulating tumor cells. An adenovirus type 5/3 for detecting circulating tumor cells, according to the present disclosure, comprises a knob domain of adenovirus type 3 and thus targets DSG2, and thus can also detect EMT circulating tumor cells, has excellent viability in the blood, and can accurately detect and isolate circulating tumor cells from more carcinomas, and thus enables early diagnosis of cancer and analysis of the type and stage of primary cancer, and can predict cancer prognosis, monitor cancer progression and analyze drug response and therapeutic effects through the genetic analysis of isolated circulating tumor cells, and therefore can be effectively used in personalized treatment.

### TECHNICAL FIELD

The present disclosure relates to a chimeric adenovirus for detecting circulating tumor cells.

### BACKGROUND ART

In 2002, the number of cancer (malignant neoplasm) deaths in the Republic of Korea was 62,887, which is 25.5% (29.6% of male deaths and 20.5% of female deaths) among a total of 246,515 deaths in the Republic of Korea (512 deaths per 100,000 population). Cancer is the number one cause of death (130.7 deaths per 100,000 population). Lung, stomach, lung, colon and pancreatic cancers predominate in order of mortality rate, while deaths from these top five cancers account for about 70% of all cancer deaths. In addition, the major causes of cancer deaths in male are lung, stomach, lung and colon cancers, while the deaths from these four major cancers (28,147) account for 70% of all male cancer deaths (40,177). The major causes of cancer deaths in female are gastric, lung, lung, colon and pancreatic cancers, while the deaths from these five cancers (13,630) account for 60% of all female cancer deaths (22,710). There are many different types of cancers currently known, reaching several dozen, and cancers are generally classified according to the tissue of origin. Cancers grow very rapidly, and invade nearby tissue, leading to metastasis, and thus may threaten life. The types of cancer include cerebrospinal tumor, head and neck cancer, lung cancer, breast cancer, thymoma, esophagus cancer, gastric cancer, colon cancer, lung cancer, pancreatic cancer, biliary tract cancer, kidney cancer, bladder cancer, lung cancer, testicular cancer, germ cell tumor, ovarian cancer, cervical cancer, endometrial cancer, lymphoma, acute leukemia, chronic leukemia, multiple myeloma, sarcoma, malignant melanoma, skin cancer, etc. Cancer may be also divided further by other classifications according to pathogenesis or morphology.

Cancer metastasis develops when cancer cells from a primary focus spread systemically through blood vessels or lymphatic vessels, and then some of the cells are engrafted to and proliferate in organs in other sites. This occurs due to circulating tumor cells (CTCs) that circulate in blood. It has been reported that the number of CTCs in the blood correlates with cancer metastasis and prognosis, so that counting the number of CTCs is useful in predicting prognoses and therapeutic effects of metastatic cancer, such as metastatic breast cancer. Hence, various studies have recently been conducted to use the CTCs as a representative marker for cancer diagnosis. The test method for detecting the CTCs is called liquid biopsy. Unlike tissue biopsy, which is used for conventional cancer diagnosis, the liquid biopsy utilizes biological fluids such as blood, urine, and saliva. Tissue biopsies have the disadvantage of being infrequent and burdensome to patients. The liquid biopsy may diagnose cancer early using a relatively simple method, thus gaining attention as a new alternative. However, unlike the billions of red blood cells and millions of white blood cells in the same amount of blood, CTCs exist in numbers of less than a few dozen, making detection very difficult. Accordingly, a technology is needed to accurately find and separate CTCs, which exist in extremely small quantities, at a ratio of one out of 109 red blood cells in blood, without cell loss.

In modern medical science, for the same disease, uniform treatment has been carried out by prescribing the same medicine within an established dosage range regardless of each individual's genetic traits. However, through various recent genome projects, a foundation has been established to understand human diseases at the genetic level, and accordingly, an era of personalized medical science is coming, in which disease diagnosis, treatment, and prevention are tailored to each individual by comparing each patient's genetic traits and drug responses, rather than providing universal treatment tailored to each patient with a different genetic environment. In this connection, in order to identify individual differences, it is necessary to know the individual's genetic traits. To this end, it is necessary to first collect the individual's body tissues in order to analyze the individual's genetic traits. However, in the past, there was no choice but to collect biological tissue using invasive tissue biopsy, which involves making a hole in the body to collect biological tissue (especially diseased tissue such as cancerous tissue). In addition to being costly, this method requires an invasive instrument to be placed inside the body of a patient, which may be painful for the patient during a collection process. In addition, in the case of cancer cells, the invasive process may be dangerous for a patient due to the need to collect cancer cells from the bone marrow. However, in the case of collecting the CTCs, it is simple and safe because only the patient's blood needs to be collected, so no invasive devices are required to enter the body of the patient. In addition, since blood collection may be done in a short period of time, an amount of time is saved. Moreover, the entire process of selecting the right anticancer drug for each individual based on the results of the anticancer drug reaction is shortened, so there is also an economic benefit.

The adenovirus genome is a linear double-stranded DNA of about 36 kb in size, and in particular, each terminus includes an inverted repeat sequence (ITR), and is configured of an encapsidation sequence (psi: ψ), an early gene, and a late gene. The early genes are included in E1, E2, E3, and E4 regions, and in particular, the E1 gene of adenovirus is the first gene expressed after infection of a target cell and consists of two transcription units, namely ElA and ElB. The protein expressed by the ElA gene actively progresses the cell cycle within infected cells and promotes transcription of the ElB gene and other early genes, thereby regulating viral replication and growth.

However, in cells infected with adenovirus, excessive cell cycle progression is inhibited or cell apoptosis is induced as a defense mechanism for prevention thereof (Shenk, T. Adenviridae: the viruses and their replication 3rd ed. New York: Lippincott-Raven 2111-2148 (1996) and Shenk, T. et al. Adv Cancer Res 57:47-85 (1991)). The ElB gene encodes the EIB 19 kDa protein and the EIB 55 kDa protein. Among these, the E1B 19 kDa protein plays a role in inhibiting apoptosis induced by the ElA protein and is similar in nucleotide sequence and function to Bcl-2 (Imazu, T. et al. Oncogene 18:4523-4529 (1999)). In addition, the EIB 55 kDa protein binds to p53 protein and inhibits cell apoptosis and various mechanisms induced by p53 protein, and functions to transport mRNAs of adenovirus into the cytoplasm to promote protein synthesis (Babiss, L. et al. Mol Cell Biol 5:2552-2558 (1985) and Leppard, K. et al. EMBO J 8:2329-2336 (1989)).

### DISCLOSURE

### Technical Problem

An aspect of the present disclosure is directed to providing an adenovirus for detecting circulating tumor cells (CTCs).

In addition, an aspect of the present disclosure is directed to providing a composition for detecting the CTCs.

In addition, an aspect of the present disclosure is directed to providing a method for detecting the CTCs.

In addition, an aspect of the present disclosure is directed to providing a method for providing information necessary for diagnosis of primary cancer.

In addition, an aspect of the present disclosure is directed to providing a method for providing information for personalized treatment.

In addition, an aspect of the present disclosure is directed to providing a method for selecting a personalized anticancer drug.

In addition, an aspect of the present disclosure is directed to providing a use of adenovirus for detecting the CTCs.

In addition, an aspect of the present disclosure is directed to providing a use of adenovirus for diagnosing primary cancer.

In addition, an aspect of the present disclosure is directed to providing a method for diagnosing primary cancer using the adenovirus for detecting the CTCs.

### Technical Solution

An embodiment of the present disclosure provides an adenovirus for detecting circulating tumor cells (CTCs).

In addition, an embodiment of the present disclosure provides a composition for detecting the CTCs, in which the composition includes a peptide or antibody that specifically binds to DSG2, a nucleic acid encoding the same, or a vector including the nucleic acid.

In addition, an embodiment of the present disclosure provides a method for detecting the CTCs.

In addition, an embodiment of the present disclosure provides a method for providing information necessary for diagnosis of primary cancer.

In addition, an embodiment of the present disclosure provides a method for providing information for personalized treatment.

In addition, an embodiment of the present disclosure provides a method for selecting a personalized anticancer drug.

In addition, an embodiment of the present disclosure provides a use of adenovirus for detecting the CTCs.

In addition, an embodiment of the present disclosure provides a use of adenovirus for diagnosing primary cancer.

In addition, an embodiment of the present disclosure provides a method for diagnosing primary cancer using the adenovirus for detecting the CTCs.

### Advantageous Effects

An adenovirus for detecting circulating tumor cells according to an embodiment of the present disclosure includes a knob domain protein of adenovirus type 3 and thus targets DSG2, and thus can also detect EMT circulating tumor cells, has excellent viability in the blood, and can accurately detect and isolate circulating tumor cells from more carcinomas, and thus enables early diagnosis of cancer and analysis of the type and stage of primary cancer, and can predict cancer prognosis, monitor cancer progression and analyze drug response and therapeutic effects through the genetic analysis of isolated circulating tumor cells, and therefore can be effectively used in personalized treatment.

### DESCRIPTION OF DRAWINGS

FIG. 1 is a diagram illustrating the production and identification of an adenovirus for isolation/detection of CTCs:

A: Schematic diagram of recombinant adenovirus serotype 5/3 encoding hTERT and GFP:

B: Schematic diagram illustrating a process for detecting the CTCs:

C: Fluorescence images identifying the detection ability of EpCAM (+) CTC and EpCAM (−) CTC (white arrows: CTCs); and

D: Sensitivity and specificity of CTC detection of Ad5/3.

FIG. 2 is a diagram comparing the expression levels of GFP after infection of various cancer cell lines with Ad5/3:

A: Images of A549, H460, and SK-OV-3 cell lines 24 and 48 hours after virus infection;

B: Pixel-based GFP intensity 24 and 48 hours after virus infection; and C: Changes in the percentage of GFP-positive cells 24 and 48 hours after virus infection.

FIG. 3 is a diagram identifying the expression levels of molecules that may be involved in the infection and replication of Ad5/3:
A: Protein expression levels of viral receptors DSG2, CD46, CAR and enzyme hTERT upon infection with Ad5/3 in each cancer cell line;
B: Protein expression level of DSG2 upon infection with Ad5/3 in each cancer cell line;
C: Protein expression level of hTERT upon infection with Ad5/3 in each cancer cell line;
D and E: Pearson correlation between infection rate and protein expression levels of DSG2 24 hours (D) and 48 hours (E) after Ad5/3 infection;
F and G: Pearson correlation between infection rate and protein expression levels of hTERT 24 hours (F) and 48 hours (G) after Ad5/3 infection;
H: GFP expression levels 24 hours after Ad5/3 infection in DSG2 knockdown HeLa cells; and
I: GFP expression levels 24 hours after Ad5/3 infection in DU145 cells overexpressing DSG2.

FIG. 4 is a diagram clinically identifying the number of CTCs before and after surgery in renal cell carcinoma (RCC) and prostate cancer (PCa) patients using Ad5/3 (each color line represents one patient):

A: Image of patient blood sample collection:

B: Number of CTCs before and after surgery in RCC patients:

C: Number of CTCs before and after surgery in patients with localized RCC (n=4) and patients with locally advanced RCC (n=9);

D: Number of CTCs before and after surgery in patients with nonmetastatic RCC (n=7) and patients with metastatic RCC (n=6):

E: Number of CTCs before and after surgery in patients with prostate cancer (n=20); and

F: Preoperative and postoperative prostate-specific antigen (PSA) levels in patients with prostate cancer (n=11).

FIG. 5 is a diagram illustrating the detection of somatic mutations in CTCs isolated using Ad5/3 and the identification that the detected CTCs are derived from primary cancer through gene copy number characteristics:

A: Process of forming a genetic library after selecting and isolating a single CTC and analyzing the entire genetic sequence:

B: Process of analyzing sequenced genome:

C: Number of somatic mutations present in the primary tumor and CTCs of each patient; and

D and E: Gene copy number variations identified on chromosomes 1 and 8 derived from primary tumor (D) and CTCs (E) of each patient.

FIG. 6 is a diagram illustrating the process of collecting a blood sample from a patient, infecting the same with Ad5/3 of an embodiment of the present disclosure to specifically isolate CTCs, and analyzing the genetic information of the isolated CTCs.

[Best Modes of the Invention]

Hereinafter, the present disclosure will be described in detail by way of embodiments of the present disclosure with reference to the accompanying drawings. However, the following examples are provided by way of illustration of the present disclosure. When it is determined that the specific description of known techniques or configuration well known to those skilled in the art unnecessarily obscure the gist of the present disclosure, the detailed description therefor may be omitted, and the present disclosure is not limited thereto. The present disclosure allows various modifications and applications within the description of the claims to be described later and the scope of equivalents interpreted therefrom.

Further, terminologies used herein are terms used to properly represent preferred embodiments of the present disclosure. It may vary depending on the intent of users or operators, or custom in the art to which the present disclosure belongs. Accordingly, the definitions of these terms should be based on the contents throughout this specification. In the entire specification, when a part is referred to as “comprising” a component, it means that it may further include other components without excluding other components unless specifically described otherwise.

Unless otherwise defined, all the technical terms used herein are used with the same meaning as commonly understood by a person skilled in the art in the field related to the present disclosure. In addition, preferred methods or samples are described herein, but those similar or equivalent thereto are incorporated in the scope of the present disclosure. The contents of all the publications disclosed as references herein are incorporated in the present disclosure.

In one aspect, an embodiment of the present disclosure relates to an adenovirus for detecting circulating tumor cells (CTCs), in which the adenovirus includes: a human telomere promoter (hTERT) operably linked to ElA and ElB of an endogenous gene of the adenovirus: a marker gene inserted into an E3 region of the endogenous gene of the adenovirus; and a knob domain protein of adenovirus type 3.

In one embodiment, the adenovirus may be produced by expressing in a cell a recombinant adenovirus vector including a nucleic acid encoding: the human telomere promoter (hTERT) operably linked to the ElA and EIB of the endogenous gene of the adenovirus: the marker gene inserted into the E3 region of the endogenous gene of the adenovirus; and a knob domain of the adenovirus type 3, in which the adenovirus vector may include a nucleotide sequence represented by SEQ ID NO: 15.

In one embodiment, the adenovirus may be a recombinant adenovirus vector including a knob domain protein of adenovirus type 3.

In one embodiment, the knob domain protein of adenovirus type 3 may bind to desmoglein-2 (DSG2), and the DSG2 may include an amino acid sequence represented by SEQ ID NO: 1 and may be encoded by a nucleic acid represented by SEQ ID NO: 2 or NM_001943.

In one embodiment, the adenovirus (Ad) may be a group C adenovirus.

In one embodiment, the adenovirus may include a fiber including a tail domain of adenovirus type 5, a shaft domain of adenovirus type 5, and a knob domain of adenovirus type 3, and may be a chimeric adenovirus (Ad 5/3) including a fiber in which a fiber knob domain of adenovirus 5 virus is genetically replaced with the corresponding domain of adenovirus-3, thereby sequentially including the tail domain of Ad5, the shaft domain of Ad5, and the knob domain of Ad3.

In one embodiment, the tail domain of Ad5 may include an amino acid sequence represented by SEQ ID NO: 3, the shaft domain of Ad5 may include an amino acid sequence represented by SEQ ID NO: 4, and the knob domain of Ad3 may include an amino acid sequence represented by SEQ ID NO: 5.

In one embodiment, the fiber of Ad 5/3 may include an amino acid sequence represented by SEQ ID NO: 6.

In one embodiment, the tail domain of Ad5 may be encoded by a nucleotide sequence represented by SEQ ID NO: 7, the shaft domain of Ad5 may be encoded by a nucleotide sequence represented by SEQ ID NO: 8, and the knob domain of Ad3 may be encoded by a nucleotide sequence represented by SEQ ID NO: 9.

In one embodiment, the fiber of Ad 5/3 may be encoded by a nucleotide sequence represented by SEQ ID NO: 10.

In one embodiment, the adenovirus has a structure of 5′ITR-C1-C₂-C3-C4-C5-3′ITR, in which: the C1 may include ElA and ElB operably linked to the hTERT promoter, include only ElA operably linked to the hTERT promoter, include only E1B operably linked to the hTERT promoter, or include only the hTERT promoter; the C2 may optionally include E2B-L1-L2-L3-E2a-L4; the C3 may include E3 including a marker gene; the C4 may optionally include L5; and the C5 may optionally include E4.

In one embodiment, hTERT may be encoded prior to an E1 region driving expression of the ElA and ElB genes linked to an internal ribosome entry site (IRES).

In one embodiment, the marker gene may be inserted into the E3 region under a cytomegalovirus (CMV) promoter of the adenovirus vector.

In one embodiment, the hTERT promoter may include a nucleotide sequence represented by SEQ ID NO: 11, the ElA may include a nucleotide sequence represented by SEQ ID NO: 12, and the ElB may include a nucleotide sequence represented by SEQ ID NO: 13.

In one embodiment, an IRES sequence may additionally be included between the ElA and the ElB, in which the IRES may include a nucleotide sequence represented by SEQ ID NO: 14.

In one embodiment, the marker may be a fluorescent substance, a probe or a tag. The marker may be any chemical moiety that may be detected by any method known in the art. Examples of detectable makers may include any moiety that may be detected by spectroscopy, photochemistry, or any biochemical, immunochemical, or chemical method. An appropriate method for marking a nucleic acid probe may be selected by considering the type and the location of the marker and the probe.

Examples of markers may include enzyme, enzyme substrate, radio-isotope, fluorescent dye, chromophores, chemiluminescent label, electrochemical luminescent label, ligand having a specific binding partner, and other markers capable of reacting with each other to increase, modify, or decrease the intensity of a detection signal.

In one embodiment, the marker may be a fluorescent substance, and the fluorescent substance may be green fluorescent protein (GFP), enhanced green fluorescent protein (EGFP), or modified red fluorescent protein (mRFP).

In one embodiment, the circulating tumor cells may be circulating tumor cells in the blood.

As used herein, the terms “specifically binding” or “specifically recognized” have the same meaning as commonly known to those skilled in the art, and mean that an antigen and an antibody specifically interact with each other to lead to an immunological reaction.

As used herein, the term “probe” refers to a nucleic acid fragment, such as RNA or DNA, which may bind specifically to mRNA, and ranges from several nucleotides to several hundred nucleotides in length. The probe may be labeled to determine the presence or absence of a specific mRNA. The probe may be constructed in the form of an oligonucleotide probe, a single-stranded DNA probe, a double-stranded DNA probe, an RNA probe or the like. In an embodiment of the present disclosure, hybridization is performed using probes complementary to the AFP, HMMR, NXPH4, PITX1, THBS4 and/or UBE2T genes, and the level of gene expression may be diagnosed through hybridization. The selection of an appropriate probe and hybridization conditions may be modified based on those commonly known in the art, and are not specifically limited thereto in an embodiment of the present disclosure.

The primers or probes of an embodiment of the present disclosure may be synthesized chemically using a phosphoramidite solid support method or other well-known methods. The nucleic acid sequences may also be modified using many methods known in the art. Non-limiting examples of these modifications include methylation, capping, substitution with one or more homologues of natural nucleotides, and modification between nucleotides, such as modification with uncharged connection bodies (for example, methyl phosphonate, phosphotriester, phosphoramidite, carbamate, etc.) or charged connection bodies (for example, phosphorothioate, phosphorodithioate, etc.).

In an embodiment of the present disclosure, suitable conditions for hybridizing a probe with a cDNA molecule may be determined in a series of processes via an optimization procedure. This procedure is performed in a series of procedures by a person skilled in the art to establish a protocol for use in a laboratory. For example, conditions such as temperature, concentration of components, hybridization and washing time, buffer components and pH and ionic strength thereof may depend on various factors such as the probe length and the GC amount and target nucleotide sequence. Detailed conditions for hybridization may be disclosed in Joseph Sambrook, et al., Molecular Cloning, A Laboratory Manual, Cold Spring Harbor Laboratory Press, Cold Spring Harbor, N. Y. (2001); and M. L. M. Anderson, Nucleic Acid Hybridization, Springer-Verlag New York Inc. N. Y. (1999). For example, among the stringent conditions, high stringency conditions are as follows: hybridization in 0.5 M NaHPO4, 7% sodium dodecyl sulfate (SDS), 1 mM EDTA and at 65° C., and washing at 68° C. in 0.1×SSC (standard saline citrate)/0.1% SDS. Alternatively, high stringency conditions are as follows: washing at 48° C. in 6×SSC/0.05% sodium pyrophosphate. Low stringency conditions are as follows: washing at 42° C. in 0.2×SSC/0.1% SDS, for example.

The term “for detecting circulating tumor cells” as used herein may be used instead of or interchangeably with “for isolating circulating tumor cells” and “for identifying circulating tumor cells.”

The terms “detect”, “detecting” or “detection” as used herein may describe the general practice of finding or identifying or specifically observing a detectably labeled composition.

In one aspect, an embodiment of the present disclosure relates to a method for producing an adenovirus for detecting circulating tumor cells, in which the method includes: expressing in a cell a recombinant adenovirus vector including a nucleic acid encoding a human telomere promoter (hTERT) operably linked to ElA and EIB of an endogenous gene of the adenovirus, a marker gene inserted into an E3 region of the endogenous gene of the adenovirus, and a knob domain protein of adenovirus type 3; and purifying the adenovirus from the cell.

In one aspect, an embodiment of the present disclosure relates to a composition for detecting circulating tumor cells, in which the composition includes the adenovirus or adenovirus vector of an embodiment of the present disclosure.

In one aspect, an embodiment of the present disclosure relates to a kit for detecting circulating tumor cells including the composition for detecting the circulating tumor cells of an embodiment of the present disclosure.

In one aspect, an embodiment of the present disclosure relates to a method for detecting circulating tumor cells, in which the method includes treating a sample isolated from a subject with the composition for detecting the circulating tumor cells of an embodiment of the present disclosure.

In one embodiment, the sample may be selected from the group consisting of blood, serum, plasma, amniotic fluid, saliva, ascites, bone marrow, tears, bile, lung lavage fluid, cerebrospinal fluid, pleural effusion, synovial fluid, lymph, semen, urine, or a solution obtained by extracting proteins from a tissue biopsy or cells, most preferably blood.

In one embodiment, the method may further include determining that cells expressing a marker gene in the sample are the circulating tumor cells.

The method for detecting the circulating tumor cells of an embodiment of the present disclosure includes a method for detecting and isolating and identifying the circulating tumor cells present in the blood of a subject.

In one aspect, an embodiment of the present disclosure relates to a method for providing information necessary for diagnosis of primary cancer, in which the method includes: 1) treating a sample isolated from a subject with the composition for detecting the circulating tumor cells of an embodiment of the present disclosure: 2) isolating cells expressing a marker gene in the sample: 3) culturing the isolated cells; and 4) determining the primary cancer.

In one embodiment, the primary cancer may be at least any one selected from the group consisting of colon cancer, breast cancer, uterine cancer, cervical cancer, ovarian cancer, prostate cancer, brain tumor, head and neck cancer, melanoma, myeloma, leukemia, lymphoma, stomach cancer, lung cancer, pancreatic cancer, non-small cell lung cancer, liver cancer, esophageal cancer, small intestine cancer, anal cancer, fallopian tube carcinoma, endometrial carcinoma, vaginal carcinoma, vulvar carcinoma, Hodgkin's disease, bladder cancer, kidney cancer, ureteral cancer, renal cell carcinoma, renal pelvic carcinoma, bone cancer, skin cancer, head cancer, neck cancer, cutaneous melanoma, intraocular melanoma, endocrine cancer, thyroid cancer, parathyroid cancer, adrenal cancer, soft tissue sarcoma, urethral cancer, penile cancer, central nervous system (CNS) tumor, primary CNS lymphoma, spinal cord tumor, glioblastoma multiforme, and pituitary adenoma.

In one aspect, an embodiment of the present disclosure relates to a method for providing information for personalized treatment, in which the method includes: 1) treating a sample isolated from a subject with the composition for detecting the circulating tumor cells of an embodiment of the present disclosure: 2) isolating cells expressing a marker gene in the sample; and 3) performing genome sequencing of the isolated cells to detect and analyze genetic variations.

In one embodiment, the genome sequencing may be performed by next-generation sequencing (NGS), and is more preferably performed by whole genome sequencing (WGS).

In one embodiment, the method may further include setting a gene in which a detected genetic variation has occurred as a therapeutic target for the subject.

In one embodiment, the information for personalized treatment may be cancer diagnosis, cancer prognosis prediction, cancer progression monitoring, drug response, therapeutic effects, or a combination thereof.

The term “subject” or “individual” as used herein means any animal, including a human, monkey, cow, horse, sheep, pig, chicken, turkey, quail, cat, dog, mouse, rat, rabbit or guinea pig, which has developed or may develop cancer.

In one aspect, an embodiment of the present disclosure relates to a method for selecting a personalized anticancer drug, in which the method includes: 1) treating a sample isolated from a subject with the composition for detecting the circulating tumor cells of an embodiment of the present disclosure: 2) isolating cells expressing a marker gene in the sample: 3) culturing the isolated cells: 4) reacting the cultured cells with a candidate anticancer drug: 5) analyzing a candidate anticancer drug response; and 6) selecting a personalized anticancer drug utilizing the analyzed candidate anticancer drug response information.

In one aspect, an embodiment of the present disclosure relates to a use of an adenovirus for detecting circulating tumor cells, the adenovirus including: a human telomere promoter operably linked to ElA and EIB of an endogenous gene of the adenovirus: a marker gene inserted into an E3 region of the endogenous gene of the adenovirus; and a knob domain protein of adenovirus type 3.

In one aspect, an embodiment of the present disclosure relates to a use of an adenovirus for diagnosing primary cancer, the adenovirus including: a human telomere promoter operably linked to ElA and ElB of an endogenous gene of the adenovirus; a marker gene inserted into an E3 region of the endogenous gene of the adenovirus; and a knob domain protein of adenovirus type 3.

In one aspect, an embodiment of the present disclosure relates to a method for diagnosing primary cancer using an adenovirus for detecting circulating tumor cells, the adenovirus including: a human telomere promoter operably linked to E1 A and E1B of an endogenous gene of the adenovirus; a marker gene inserted into an E3 region of the endogenous gene of the adenovirus; and a knob domain protein of adenovirus type 3.

In one embodiment, the primary cancer may be at least any one selected from the group consisting of colon cancer, breast cancer, uterine cancer, cervical cancer, ovarian cancer, prostate cancer, brain tumor, head and neck cancer, melanoma, myeloma, leukemia, lymphoma, stomach cancer, lung cancer, pancreatic cancer, non-small cell lung cancer, liver cancer, esophageal cancer, small intestine cancer, anal cancer, fallopian tube carcinoma, endometrial carcinoma, vaginal carcinoma, vulvar carcinoma, Hodgkin's disease, bladder cancer, kidney cancer, ureteral cancer, renal cell carcinoma, renal pelvic carcinoma, bone cancer, skin cancer, head cancer, neck cancer, cutaneous melanoma, intraocular melanoma, endocrine cancer, thyroid cancer, parathyroid cancer, adrenal cancer, soft tissue sarcoma, urethral cancer, penile cancer, central nervous system (CNS) tumor, primary CNS lymphoma, spinal cord tumor, glioblastoma multiforme, and pituitary adenoma.

### Modes of the Invention

The present disclosure is explained in more detail through the following examples. However, the following examples are only intended to concretize the contents of the present disclosure, and the present disclosure is not limited thereby.

### Example 1. Design and Preparation of Adenovirus 5/3 (Ad 5/3) Chimeric Vector for CTC Detection

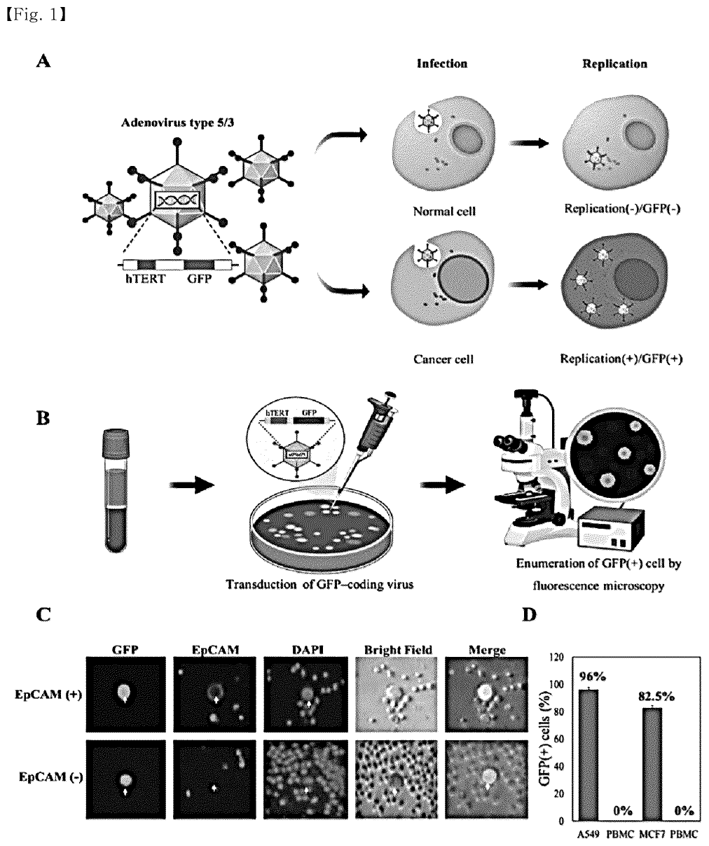

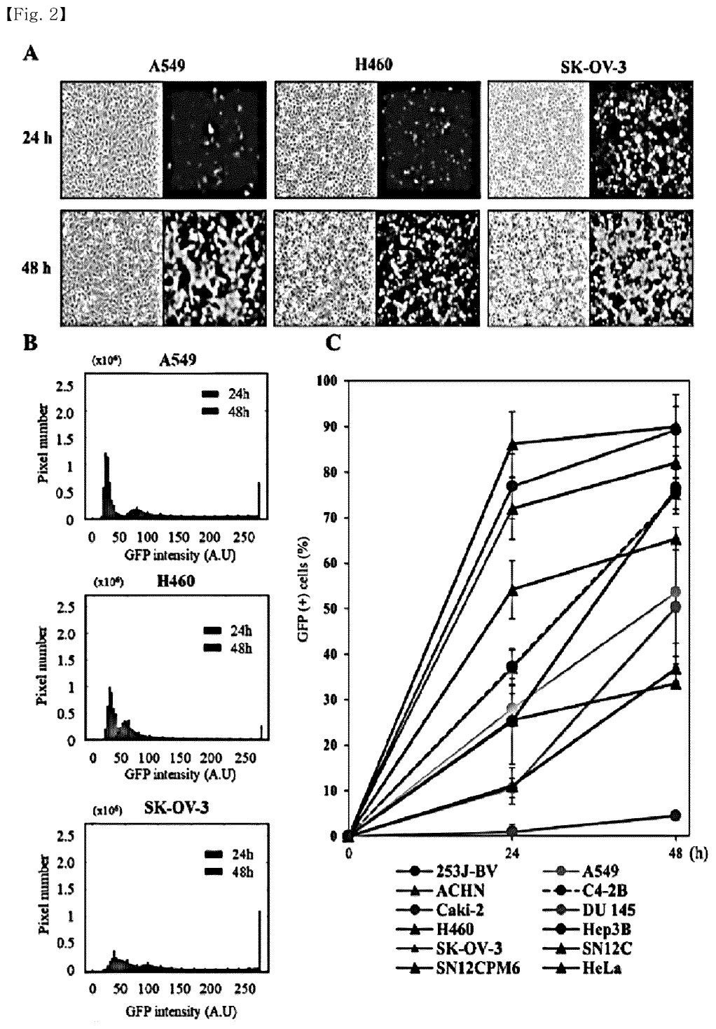

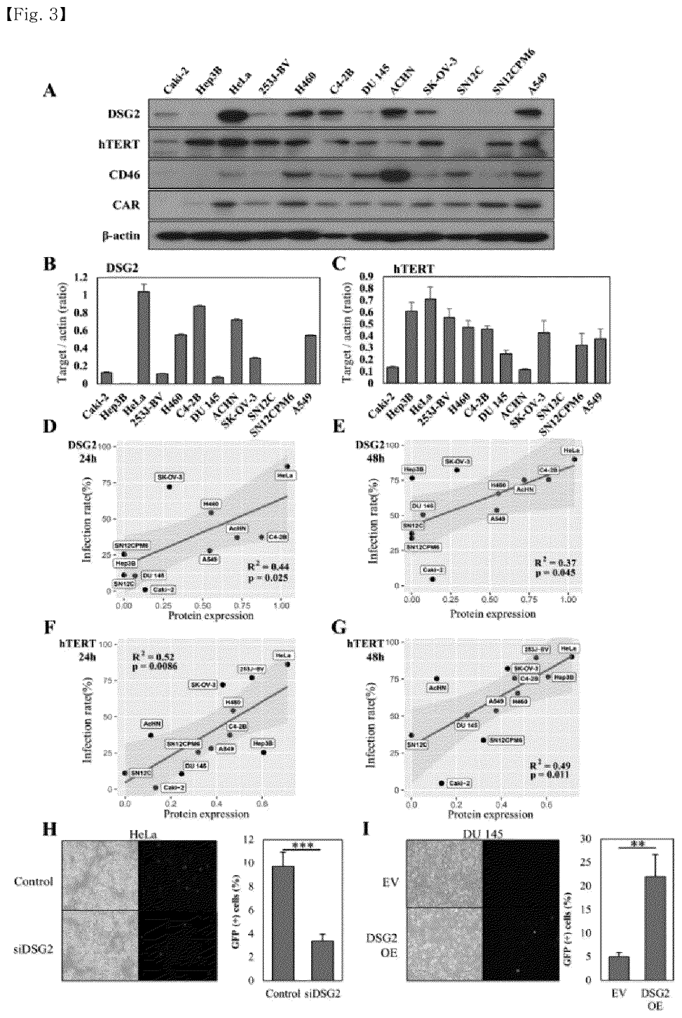

A coxsackievirus and adenovirus receptor (CAR), a binding receptor for adenovirus serotype 5, is known to be down-regulated in advanced tumors, and adenovirus serotype 3 fiber uses DSG2 (desmoglein-2) as its main receptor. Accordingly, an adenovirus 5/3 (Ad 5/3) chimera (in other words, an adenovirus having a fiber sequentially including a tail domain of Ad5, a shaft domain of Ad5, and a knob domain of Ad3) including the knob domain of adenovirus 3 in the fiber of adenovirus serotype 5 was designed and prepared to enable specific infection of advanced tumors and solid tumors by targeting both CAR and DSG2. Specifically, the viral vector system Ad5/3: pAd1127, pAd1128, pAd1129, and pAd1130 were purchased from DO260 Inc. (ID, USA), and the viral backbone was prepared using Cosmid construction kit-2 according to the protocol (OD260). In addition, the virus was designed to encode before an E1 region driving the expression of the ElA and ElB genes linked to the internal ribosome entry site (IRES) so that GFP is expressed under human telomerase reverse transcriptase (hTERT) expression conditions, and to stop the E1 region of the virus, which is essential for the replication of Ad 5/3, in the absence of the expression of the hTERT-specific binding transcription factor. In addition, the green fluorescent protein (GFP) gene for fluorescence expression was inserted into the E3 region under the cytomegalovirus (CMV) promoter of the Ad 5/3 genome. Pac-I (NEB, MA, USA)-cleaved full-length viral genome was transfected into 293A cells (Invitrogen, CA, USA) using the CalPhos™ Mammalian Transfection Kit (Clontech, Ltd.) to produce a recombinant virus, and was amplified and purified using the Adeno-XTM Mega Purification Kit (Clontech). The purified virus was stored at-80° C., and the infectious virus titer was identified and determined in human embryonic kidney (HEK) 293 cells (ATCC) using the Adeno-XTM Rapid Titer Kit (Clontech). This genetically modified virus, Ad 5/3, may infect both normal cells and cancer cells. However, due to the specificity of the promoter, virus replication and GFP expression may occur only in cancer cells (FIG. 1A). Using this characteristic, cells expressing GFP in patient samples, in other words, CTCs, may be observed using a fluorescence microscope (FIG. 1B).

### Example 2. Identification of CTC-Specific Detection Ability of Ad 5/3

In order to identify the specific infection of CTC (circulating tumor cells) and the sensitivity and specificity of CTC detection of the virus Ad 5/3 prepared in Example 1, 100 each of the cancer cell lines of A549 cell line and MCF7 cell line and the blood cells of peripheral blood monocytic cells (PBMC) were dispensed, and then treated with 10 MOI (multiplicity of infection) of adenovirus Ad 5/3 and incubated for 24 hours. Thereafter, fluorescence was observed using a Nikon DIAPHOT 300 (Nikon, Japan) with an illumination device to check GFP-positive cells and EpCAM-positive cells (stained with anti-EpCAM (Cell Signaling Technology Inc., CA, USA)), and the infection rate was calculated by dividing the number of GFP-expressing cells by the total number of cells. Each cell line was counted using ImageJ (v2.0) software. Each experiment was repeated five times, GFP-positive cells and EpCAM-positive cells were identified under a fluorescence microscope, and the percentage of GFP-positive cells was counted to calculate the sensitivity and specificity of the virus (repeated five times).

As a result, it was identified that the virus of an embodiment of the present disclosure was able to detect not only EpCAM (+) CTCs but also EpCAM (−) CTCs, thereby infecting CTCs and expressing GFP regardless of whether EpCAM was expressed in the CTCs (FIG. 1C). Furthermore, the sensitivity and specificity of GFP-encoding Ad 5/3-based CTC detection were identified by detecting 96% of A549 cells and 82.5% of MCF7 cells, whereas no GFP expression was observed in PBMCs (FIG. 1D).

Thus, it was identified that the Ad 5/3 of an embodiment of the present disclosure may selectively detect CTCs among PBMCs and that both EpCAM (+) and EpCAM (−) CTCs were able to be detected.

### Example 3. Identification of Detection Ability of Ad 5/3 for Various Types of CTCs

In order to identify the diagnostic efficacy of various types of CTCs of the adenovirus (Ad 5/3) prepared in Example 1 above, the GFP expression levels in various cancer cell lines were identified and compared. Specifically, 12 cancer cell lines of 253J-BV (human bladder cancer), ACHN (papillary renal cancer), Caki-2 (clear cell carcinoma), SN12C (human renal cancer), SN12PM6 (human kidney cancer) (Korean Cell Line Bank, Seoul, Korea), Hep3B (hepatocellular carcinoma), A549 (lung adenocarcinoma), H460 (lung large cell carcinoma), SK-OV-3 (ovarian cancer), C4-2B (prostate cancer), HeLa (cervical cancer), and DU 145 (human prostate cancer) (ATCC: VA, USA) were each inoculated in a 6-well plate at a cell concentration of 3×10⁵, cultured for 24 hours, and then replaced with fresh medium containing 15 MOIs of the CTC-specific adenovirus 5/3 (Ad 5/3) of Example 1. After 24 and 48 hours of incubation, the number of GFP-positive cells was counted using a fluorescence microscope. The infection rate was calculated by dividing the number of GFP expressing cells by the total number of cells and was calculated for each cell line using ImageJ& software. To visualize GFP intensity between two time points, an analysis was performed at a pixel level, the brightness was converted to the range of 0 to 250, and the cell number was analyzed as the number of pixels.

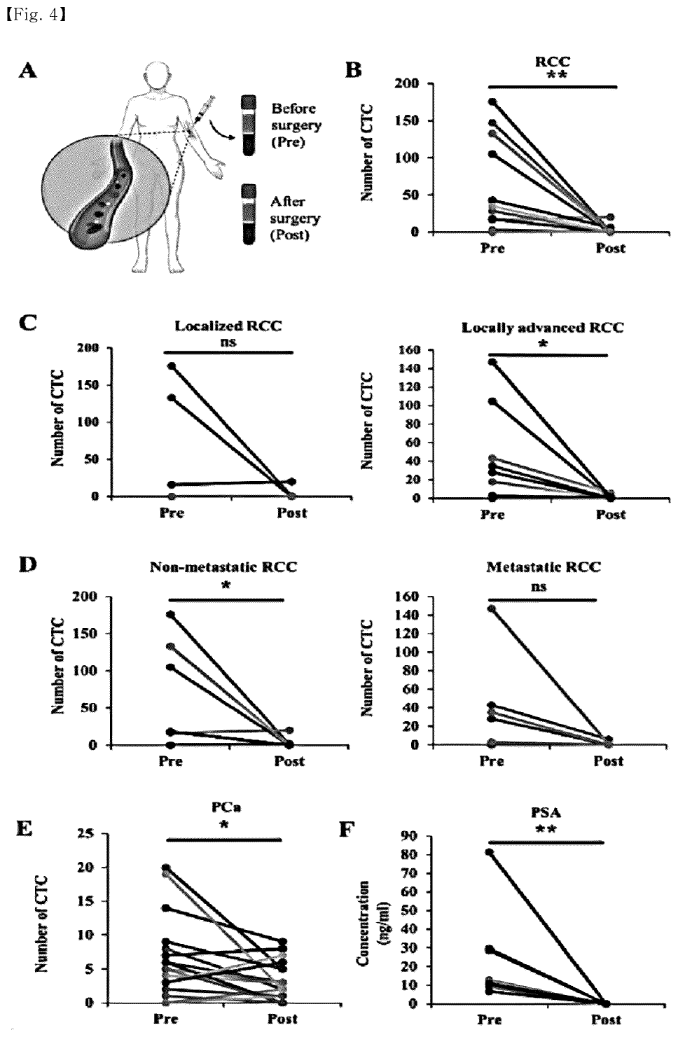

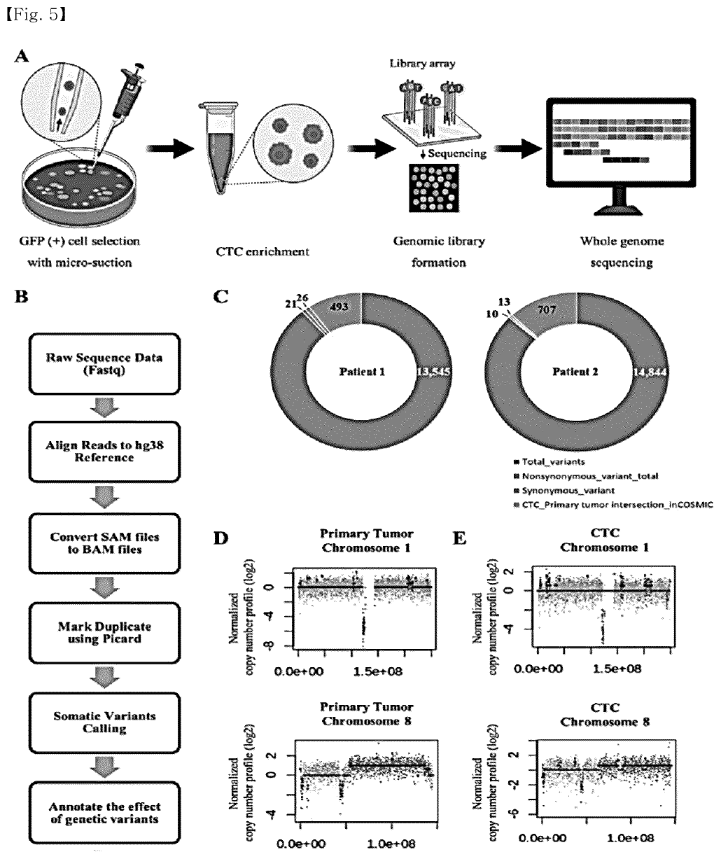

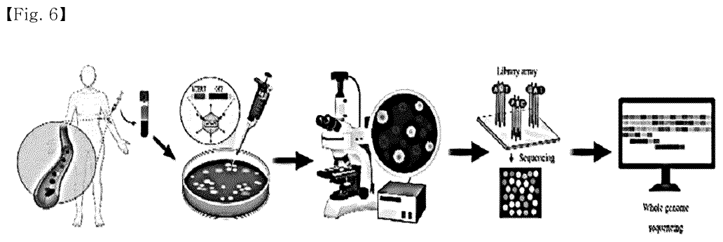

As a result, the infection rate and GFP intensity varied with incubation time (FIG. 2A), generally showing a relatively higher cell number (determined by pixel number) at 48 hours compared to 24 hours (FIG. 2B). In addition, although the infection rate tended to increase over time in all cell lines, the ratio between 24 hours and 48 hours was different for each cell line, and in particular, the initial infection rate was different. In addition, the percentage of GFP-positive cells varied between cell lines (FIG. 2C), identifying that the expression and replication rates of the viral receptor differ between cell lines.

### Example 4. Correlation between Cancer Cell Infection Rate of Ad5/3 and DSG2 4-1. Identification of Changes in Expression of Adenovirus Receptors Caused by Adenovirus Infection

The adenovirus Ad 5/3 of an embodiment of the present disclosure has an Ad5 structure in which the fiber knob of Ad3 is attached, and the fiber knob of type 3 binds to DSG2 (desmoglein-2) expressed on the cell membrane. Accordingly, the correlation between the expression levels of DSG2 and other viral receptors, such as CAR (coxsackievirus and adenovirus receptor) and CD46 (cluster of differentiation 46), and the replication-related gene hTERT (human telomerase reverse transcriptase), and the adenovirus infectivity was identified by Western blot analysis. Specifically, 12 cancer cell lines of Example 2 were infected with Ad 5/3, and lysed with RIPA buffer (Cure Bio, Korea) after 24 and 48 hours, respectively. Then, an equal volume of protein lysate was electrophoresed on an SDS-PAGE gel and transferred to a nitrocellulose membrane. The membrane was blocked with 5% w/v of skim milk (in TBST) and then reacted with primary antibodies against coxsackievirus and adenovirus receptor (1:1000 dilution, Bioss, MA, USA), CD46 (1:1000 dilution, Prosci, Inc, CA, USA), DSG2 (1:1000 dilution, Santa Cruz, TX, USA), hTERT (1:1000 dilution, Santa Cruz), and β-actin (Santa Cruz), respectively. The membrane was washed three times with TBST, reacted with a secondary antibody for 2 hours, and washed three times with TBST. Thereafter, proteins on the membrane were visualized with luminol reagent (Santa Cruz) and detected using an automatic X-ray film processor JP-33 (JPI, Korea). Western blot bands of detected DSG2 and hTERT were semi-quantitatively analyzed and normalized to B-actin levels. In addition, the correlation between the infection rates at 24 and 48 hours and the protein expression levels of DSG2 was identified using Pearson correlation analysis.

As a result, it was shown that the difference in the expression of DSG2 and hTERT between cell lines could affect the infectivity of Ad5/3, and that the protein expression level of DSG2 or hTERT affected the infectivity (FIG. 3A to FIG. 3C). In addition, as a result of analyzing the correlation between receptor expression and viral infection rate, it was shown that the correlation coefficient was relatively high in DSG2

(FIG. 3D and FIG. 3E). In addition, hTERT, the enzyme responsible for viral replication, showed a correlation with GFP expression (FIG. 3F and FIG. 3G). In addition, HeLa cell lines showed high infection rates of more than 80% at both time points (24 hours and 48 hours) assuming high expression of DSG2 and hTERT (FIG. 3B and FIG. 3C). Hep3B cell lines showed relatively low DSG2 expression level and high hTERT expression level (FIG. 3B and FIG. 3C), which seemed to be related to low GFP expression at 24 hours (25.3%) and high infection at 48 hours (76.4%). SN12CPM6 cell lines showed almost similar infection to Hep3B cell lines at 24 hours (25.5%), but its GFP was significantly expressed at 48 hours (33.5%), unlike Hep3B cell lines (76.4%). The relatively low infection at 48 hours in SN12CPM6 was inferred to be associated with low hTERT protein expression.

### 4-2. Identification of Correlation between DSG2 Expression Changes and Viral Infectivity

After artificially inducing expression suppression or overexpression of DSG2 using siRNA or an expression vector, infection by the virus of an embodiment of the present disclosure was identified using a fluorescence microscope. Specifically, for the knockdown of DSG2, 1×105 HeLa cells were dispensed in 6-well plates. After 24 hours, the negative control group (Thermofisher Scientific, Inc.) and siDSG2 (Bioneer, Korea) were transfected with Lipofectamine™ 2000 (Thermofisher Scientific, Inc.), respectively. After 48 hours, the adenovirus Ad 5/3 of an embodiment of the present disclosure was treated with 5 MOI and incubated for 24 hours. Then, fluorescence was identified under a fluorescence microscope. In addition, for overexpression of DSG2, 1×105 DU145 cell lines were dispensed in 6-well plates, and pmCherry-DSG2 (Addgene, MA, USA) and its empty vector were each transfected using Lipofectamine™ 3000 (Thermofisher Scientific, Inc.). After 24 hours, the adenovirus Ad 5/3 of an embodiment of the present disclosure was treated with 15 MOI and incubated for 24 hours. Then, fluorescence was identified under a fluorescence microscope.

As a result, downregulation of DSG2 expression in HeLa cells, in which DSG2 is expressed relatively highly, was found to decrease GFP expression due to low virus infection at an early point in time at low MOI (FIG. 3H). In contrast, when DSG2 was overexpressed in DU145 cell lines, GFP expression was higher than under the previous conditions (FIG. 31).

Thus, it was identified that DSG2 had a direct correlation with Ad5/4 virus infection into target cancer cells.

### Example 5. Clinical Application and Validation of Adenovirus for CTC Detection

In general, the number of CTCs decreases after debulking of the primary tumor. It has been reported that patients with a stable number of CTCs after surgery have a poor prognosis, and thus it may be assumed that the primary tumor is the main cause of CTCs. Accordingly, in order to verify whether the adenovirus Ad5/3 for CTC detection of an embodiment of the present disclosure is able to specifically infect and detect CTCs in actual clinical specimens before and after surgery and identify the above-mentioned changes in CTC counts, blood samples were collected from 17 renal cell carcinoma (RCC) patients and 20 prostate cancer (PCa) patients (Advanced RCC: H-1811-087-986, Metastatic RCC: H-1805-186-950, Prostate cancer: H-1607-134-777) before surgery and 30 days after surgery (interval between the two time points: 1 month) (FIG. 4A). To count the number in each collected blood sample, 9 ml of blood was transferred to a heparin-coated tube at 4° C., and the sample was mixed with red blood cell (RBC) lysis solution (Qiagen, Hilden, Germany) in a ratio of 1:3 and incubated at RT for 20 minutes. Each tube was then centrifuged at 300×g for 5 minutes at RT, and the cells remaining at the bottom of the tube were collected and washed twice with serum-free DMEM. The adenovirus of an embodiment of the present disclosure was incubated in DMEM containing 3×10⁹ VG/mL for 24 hours in a CO₂ incubator at 37° C., and then CTCs were isolated and counted from PBMCs as in the above example. The grade of cancer was assessed by the pathologic grade of each tumor cell after surgery.

As a result of identifying the number of CTCs in preoperative and postoperative samples, it was shown that the number of CTCs decreased after surgery in RCC patients (FIG. 4B). Thereafter, the T factor of the TNM staging system was used to classify the degree of cancer localization into two groups in RCC patients (localized: T1 (n=4), locally advanced: T2-3 (n=9)), and the change in the number of CTCs was identified. As a result, it was found that only patients with locally advanced RCC had a significant decrease in CTCs after surgery (FIG. 4C). In addition, as a result of classifying patients into non-metastatic and metastatic RCC patients and identifying the changes in the number of CTCs, it was shown that only non-metastatic RCC patients showed a significant decrease in CTCs after surgery (FIG. 4D). In addition, the number of CTCs was found to decrease after surgery in PCa patients (FIG. 4E), and the level of prostate-specific antigen (PSA), a marker of prostate cancer, was also found to decrease after surgery (FIG. 4F).

Thus, it was identified that the CTC detection technology using the adenovirus of an embodiment of the present disclosure was accurate enough to observe a post-surgical decrease in CTCs.

### Example 6. CTC Isolation and Analysis using Ad5/3

In order to verify whether the method of an embodiment of the present disclosure is able to be used to collect CTCs and detect somatic mutations therefrom, 2 to 3 CTCs were isolated from samples of two RCC patients using a micro-suction system with the virus Ad5/3 of an embodiment of the present disclosure, and whole genome sequencing (WGS) was performed on the CTCs and primary tumors (FIG. 5A). Specifically, CTCs expressing GFP were isolated from the PBMC layer, resuspended in serum-free medium, and single cell picking was performed using the Micro Pick and Placement system (Nepa Gene Co., Ltd, Japan). Thereafter, genomic DNA of CTCs was obtained and converted to an Illumina library using the PicoPLEX® Gold single cell DNA-Seq kit (Takara Bio Inc., CA, USA) and DNA HT Dual index kit (Takara Bio). Raw sequence data from FASTQ files with an average sequencing depth greater than 20X were obtained by Illumina Paired-End (PE) sequencing. The read data was mapped to the hg38 (human genome 38) reference genome using the Burrows-Wheeler Aligner (BWA)-MEM algorithm (0.7.17). The aligned SAM files were converted and aligned to BAM files using Samtools (v1.7), duplicate reads from each BAM file were removed using Picard (v2.23.8), and somatic variations were identified using the Mutect2 tool of GATK (v.4.1.8). The functional effects of variations were annotated using VEP (v102), and these variations were filtered by expected medium or high impact on the consequence types. COSMIC (Catalog of Somatic Mutations in Cancer: v92) was used to annotate COSV IDs for each somatic variation (FIG. 5B). The comparison of copy number variations (CNVs) between primary tumors and CTCs was evaluated using FREEC algorithms (v11.6) with breakpointThreshold 0.6 and no additional options. Since gene amplification by PCR may cause mutations, copy number characteristics between primary tumors and CTCs were identified only on chromosomes 1 and 8, representatively.

The number of somatic mutations present in both primary tumors and CTCs using Mutect2 was found to be 13,545 and 14,844 in two patients, respectively. Of these, 21 and 10 somatic nonsynonymous variations causing amino acid variations were found in primary tumors and CTCs, respectively. In addition, 493 and 707 somatic mutations present in the primary tumor and CTCs of each patient were identified by COSMIC (FIG. 5C). In addition, CNVs analysis results showed that the isolated CTCs originated from the primary cancer site (FIG. 5D and FIG. 3E).

Thus, it was identified that the CTC detection method using the adenovirus of an embodiment of the present disclosure may specifically isolate/extract only CTCs from among numerous blood cells, particularly PBMCs, and analyze their characteristics, such as mutations, using NGS (next generation sequencing), particularly WGS, and thereby predict drug response and therapeutic effects, and apply the same to personalized treatment.

1. An adenovirus for detecting circulating tumor cells (CTCs), the adenovirus comprising:
a human telomere promoter (hTERT) operably linked to E1A and E1B of an endogenous gene of the adenovirus, further comprising an IRES sequence between the E1A and the E1B;
a marker gene inserted into an E3 region of the endogenous gene of the adenovirus; and
a knob domain protein of adenovirus type 3.

2. The adenovirus of claim 1, wherein the knob domain protein of adenovirus type 3 includes an amino acid sequence represented by SEQ ID NO.: 5.

3. The adenovirus of claim 1, wherein the knob domain protein of adenovirus type 3 binds to DSG2 (desmoglein-2).

4. The adenovirus of claim 1, wherein the adenovirus includes a fiber including a tail domain of adenovirus type 5, a shaft domain of adenovirus type 5, and a knob domain of adenovirus type 3.

5. The adenovirus of claim 4, wherein the fiber includes an amino acid sequence represented by SEQ ID NO.: 6.

6. The adenovirus of claim 1, wherein the adenovirus is translated from an adenovirus vector including a nucleotide sequence represented by SEQ ID NO.: 15.

7. The adenovirus of claim 1, wherein the endogenous gene of the adenovirus has a structure of 5′ITR-C1-C₂-C3-C4-C5-3′ITR, wherein: the C1 includes E1A and E1B operably linked to the hTERT promoter, includes only ElA operably linked to the hTERT promoter, includes only E1B operably linked to the hTERT promoter, or includes only the hTERT promoter; the C2 optionally includes E2B-L1-L2-L3-E2a-L4; the C3 includes E3 includes the marker gene; the C4 optionally includes L5; and the C5 optionally includes E4.

8. (canceled)

9. The adenovirus of claim 1, wherein a marker is a fluorescent substance, a probe or a tag.

10. The adenovirus of claim 9, wherein the fluorescent substance is green fluorescent protein (GFP), enhanced green fluorescent protein (EGFP), or modified red fluorescent protein (mRFP).

11. (canceled)

12. (canceled)

13. A method for detecting circulating tumor cells, the method comprising treating a sample isolated from a subject with a composition comprising the adenovirus of claim 1.

14. (canceled)

15. (canceled)

16. A method for providing information necessary for diagnosis of primary cancer, the method comprising:
1treating a sample isolated from a subject with the composition for detecting the circulating tumor cells of claim 11;
2isolating cells expressing a marker gene in the sample;
3culturing the isolated cells; and
4determining the primary cancer.

17. The method of claim 16, wherein the primary cancer is at least any one selected from the group consisting of colon cancer, breast cancer, uterine cancer, cervical cancer, ovarian cancer, prostate cancer, brain tumor, head and neck cancer, melanoma, myeloma, leukemia, lymphoma, stomach cancer, lung cancer, pancreatic cancer, non-small cell lung cancer, liver cancer, esophageal cancer, small intestine cancer, anal cancer, fallopian tube carcinoma, endometrial carcinoma, vaginal carcinoma, vulvar carcinoma, Hodgkin's disease, bladder cancer, kidney cancer, ureteral cancer, renal cell carcinoma, renal pelvic carcinoma, bone cancer, skin cancer, head cancer, neck cancer, cutaneous melanoma, intraocular melanoma, endocrine cancer, thyroid cancer, parathyroid cancer, adrenal cancer, soft tissue sarcoma, urethral cancer, penile cancer, central nervous system (CNS) tumor, primary CNS lymphoma, spinal cord tumor, glioblastoma multiforme, and pituitary adenoma.

18. A method for providing information for personalized treatment, the method comprising:
1treating a sample isolated from a subject with the composition for detecting the circulating tumor cells of claim 11;
2isolating cells expressing a marker gene in the sample; and
3performing genome sequencing of the isolated cells to detect and analyze genetic variations.

19. The method of claim 18, wherein the genome sequencing is performed by next-generation sequencing (NGS).

20. The method of claim 18, further comprising setting a gene in which the detected genetic variation has occurred as a therapeutic target for the subject.

21. The method of claim 18, wherein the information for personalized treatment is cancer diagnosis, cancer prognosis prediction, cancer progression monitoring, drug response, therapeutic effects, or a combination thereof.

22-25. (canceled)
